(19) United States
(12) Patent Application Publication
(10) Pub. No.: US 2025/0163102 A1
(43) Pub. Date: May 22, 2025

(54) Protein Purification Methods and Systems

(71) Applicant: Genentech Inc., South San Francisco, CA (US)

(72) Inventors: Maria Nieves Lorenzo, South San Francisco, CA (US); May Lin, South San Francisco, CA (US)

(73) Assignee: Genentech Inc., South San Francisco, CA (US)

(21) Appl. No.: 19/032,546

(22) Filed: Jan. 21, 2025

Related U.S. Application Data
(63) Continuation of application No. PCT/US2023/071192, filed on Jul. 28, 2023.
(60) Provisional application No. 63/393,795, filed on Jul. 29, 2022.

(51) Int. Cl. C07K 1/36; B01D 15/20; B01D 15/34; B01D 15/38; B01D 15/42; C07K 1/22; C07K 1/34

(52) U.S. Cl. CPC C07K 1/36; B01D 15/203; B01D 15/34; B01D 15/3828; B01D 15/424; C07K 1/22; C07K 1/34

(57) ABSTRACT

The present application relates to systems and methods for purifying polypeptides, such as His-tagged polypeptides, using immobilized metal chelate affinity chromatography (IMAC). The method includes loading a composition comprising the polypeptide onto a IMAC matrix; eluting the polypeptide from the first IMAC matrix; conducting a buffer exchange to remove the elution buffer from the first eluate; loading the first eluate from the buffer exchange onto a second IMAC matrix; eluting the polypeptide from the second IMAC matrix; and optionally purifying the eluate from the second IMAC matrix using size exclusion chromatography. In some aspects, the method is fully automated.

### CROSS-REFERENCE TO RELATED APPLICATIONS

The application is a continuation of International Application No. PCT/US2023/071192, filed Jul. 28, 2023, which claims the benefit of priority of U.S. Provisional Application No. 63/393,795, filed Jul. 29, 2022, the contents of which are incorporated herein by reference in their entireties.

### FIELD

The present application relates to systems and methods for purifying polypeptides, such as His-tagged polypeptides, using immobilized metal chelate affinity chromatography (IMAC), for example using two different IMAC matrices in a high-throughput set-up. In some embodiments, the IMAC matrix comprises a chelation moiety bound to nickel.

### BACKGROUND

Drug discovery research and basic research involving polypeptides often requires reliable, high quality protein reagents, and often requires reagents that may be purified quickly, such as in a high-throughput setting. Purification of secreted his-tagged proteins generally involves concentration of the media containing the His-tagged protein, ultrafiltration to exchange the buffer, one round of immobilized metal affinity chromatography (IMAC), often a nickel-containing Ni NTA matrix, to selectively bind protein with a His-tag, and elution of the His-tagged protein from the IMAC column (FIG. 1). However, this method involves a time-intensive step of ultrafiltration in order to exchange the buffer to one that is compatible with a Ni NTA matrix, which significantly increases the time required for preparing pure protein. New systems and methods are needed to efficiently and reliably purify protein to meet reagent standards of modern drug discovery and polypeptide research.

### SUMMARY

The present disclosure relates to a novel purification method involving two different IMAC matrices for purifying His-tagged polypeptides or other polypeptides that selectively bind to a metal ion chelation matrix. Particular embodiments herein include, for example, a method for purifying a polypeptide, comprising: (a) loading a composition comprising the polypeptide onto a first immobilized metal affinity chromatography (IMAC) matrix; (b) eluting the polypeptide from the first IMAC matrix with an elution buffer to form a first eluate comprising the polypeptide; (c) conducting a buffer exchange to remove the elution buffer from the first eluate; (d) loading the first eluate from step (c) onto a second IMAC matrix; and (e) eluting the polypeptide from the second IMAC matrix with a second elution buffer to form a second eluate comprising the polypeptide. In some embodiments, the method further comprises conducting size exclusion chromatography (SEC) on the second eluate. In some cases, the method further comprises detecting the polypeptide in the first or second eluate or following SEC, optionally by electrophoresis or mass spectrometry. In some embodiments, the first IMAC matrix comprises agarose beads and a chelating ligand bound to nickel. In some embodiments, the first IMAC matrix retains nickel when exposed to cell culture media. In some embodiments, the second IMAC matrix comprises agarose beads and a nitrilotriacetic acid chelation moiety bound to nickel. In some cases, the method further comprises regenerating the first IMAC matrix and/or the second IMAC matrix by washing with a regeneration buffer. In some such cases, the regeneration buffer comprises sodium hydroxide. In some embodiments, the polypeptide comprises a polyhistidine tag (His-Tag). In some embodiments, the polypeptide is an antibody, cell receptor, intracellular protein, secreted protein, or membrane protein. In some embodiments, the polypeptide is a secreted protein. In some embodiments, the buffer exchange comprises passing the first eluate over a desalting column. In some embodiments, the first elution buffer and the second elution buffer are the same. In some cases, the composition comprises cell culture media. In some cases, the cell culture media is conditioned cell culture media. In some cases, the method further comprises washing the first IMAC matrix with a wash buffer after step (a) and before step (b). In some cases, the method further comprises washing the second IMAC matrix with a wash buffer after step (d) and before step (e). In some cases, a controller directs flow through columns that contain the first IMAC matrix and the second IMAC matrix the method is automated. In some cases, the method further comprises determining an elution time window for the first eluate and the second eluate. The disclosure herein also relates to a polypeptide which has been purified according to the methods herein.

The present disclosure also includes a system for purifying a polypeptide. In some embodiments, the system comprises: (a) an injection valve for injecting a composition containing the polypeptide into the system, wherein the injection valve is connected to a first immobilized metal affinity chromatography (IMAC) matrix and a pump to control flow of the sample through the first IMAC matrix; (b) a column valve for controlling flow path of an equilibration buffer, an elution buffer, and an SEC buffer; (c) a desalting column configured to receive flowthrough or eluate from the first IMAC matrix; (d) a second IMAC matrix configured to receive flowthrough or eluate from the desalting column; (e) a size exclusion chromatography (SEC) column configured to receive flowthrough or eluate from the second IMAC matrix; and (f) a detection device for detecting the polypeptide in the system. In some cases, the injection valve and the column valve can be automatically controlled. In some cases, the first and/or second IMAC matrix is an IMAC column. In some cases, the detection device is a UV spectrometer or mass spectrometer.

The present disclosure further comprises, inter alia, a method for purifying a His-tagged polypeptide, comprising: (a) loading a composition comprising the His-tagged polypeptide onto a first immobilized metal affinity chromatography (IMAC) matrix; (b) eluting the His-tagged polypeptide from the first IMAC matrix with an elution buffer to form a first eluate comprising the polypeptide; (c) conducting a buffer exchange to remove the elution buffer from the first eluate using a desalting column; (d) loading the first eluate from step (c) onto a second IMAC matrix, wherein the second IMAC matrix comprises agarose beads and a nitrilotriacetic acid chelation moiety bound to nickel; and (e) eluting the His-tagged polypeptide from the second IMAC matrix with a second elution buffer to form a second eluate comprising the His-tagged polypeptide. In some embodiments, the method further comprises conducting size exclusion chromatography (SEC) on the second eluate to collect purified polypeptide. In some cases, the method further comprises detecting the purified polypeptide collected from the SEC, optionally by electrophoresis or mass spectrometry. In some cases, the first IMAC matrix comprises agarose beads and a chelating ligand bound to nickel. In some cases, the first IMAC matrix retains nickel when exposed to cell culture media. In some cases, the second IMAC matrix comprises agarose beads and a nitrilotriacetic acid chelation moiety bound to nickel. The disclosure also comprises a polypeptide which has been purified according to such a method. In some embodiments, the polypeptide is a secreted polypeptide.

Additional objects and advantages will be set forth in part in the description which follows, and in part will be understood from the description, or may be learned by practice. The objects and advantages will be realized and attained by means of the elements and combinations particularly pointed out in the appended claims. It is to be understood that both the foregoing general description and the following detailed description are exemplary and explanatory only and are not restrictive of the claims.

The accompanying drawings, which are incorporated in and constitute a part of this specification, illustrate certain embodiments and together with the description, serve to further explain certain principles described herein.

### BRIEF DESCRIPTION OF THE DRAWINGS

FIG. 1 shows a flow chart for a typical, traditional purification of a secreted His-tagged polypeptide.

FIG. 2A shows elution profiles of a His-tagged polypeptide from a Ni Excel column at different concentrations of imidazole in the conditioned media.

FIG. 2B shows a gel electrophoresis panel of main fractions from the tests at 0 mM imidazole, 10 mM imidazole, and 20 mM imidazole.

FIG. 3A shows an elution profile of a His-tagged polypeptide from a Ni Excel column.

FIG. 3B shows a gel electrophoresis panel of main Ni Excel peak elution fractions from the test in FIG. 3A

FIG. 4 shows an elution profile for a two stage purification method of a His-tagged polypeptide and a magnified plot of the portion of the profile when the His-tagged polypeptide and impurities eluted, as depicted in the bottom graph.

FIG. 5A shows a flow chart of a portion of an exemplary two stage purification method herein, comprising use of a Ni Excel column to purify a polypeptide from BEVS culture media, followed by further purification on a Superdex® SEC column, and regeneration of the Ni Excel column.

FIG. 5B shows an overlay of the elution profiles for multiple runs of the same polypeptide and gel electrophoresis results from the purification of a high expressed His-tagged polypeptide in BEVS media as shown in FIG. 5A.

FIG. 6A shows an Ni Excel elution profile of a low expressed His-tagged polypeptide in BEVS media.

FIG. 6B shows gel electrophoresis results of the low expressed His-tagged polypeptide of FIG. 6A, and shows low purity of the Ni-Excel elution indicating that, while compatible with the BEVS media a Superdex® SEC column alone will not be sufficient to remove all contaminants in the Ni-Excel eluted material.

FIG. 7A shows an elution profile of a highly expressed His-tagged polypeptide in CHO media purified using a Ni Excel column without further SEC purification.

FIG. 7B shows gel electrophoresis results of eluate from the Ni Excel column loaded with high expressed His-tagged polypeptide in CHO media as shown in FIG. 7A, and shows that the Ni Excel column pulls down several impurities as well as the desired His-tagged polypeptide.

FIG. 8A shows an elution profile after SEC purification of the His-tagged polypeptide of FIGS. 7A and 7B.

FIG. 8B shows gel electrophoresis results of eluate from the SEC purification of FIG. 8A, showing that impurities are not efficiently removed by the SEC.

FIG. 9 shows an embodiment of a system for purifying a His-tagged polypeptide via a two stage process with two IMACs, in this case Ni Excel followed by NiNTA, followed by SEC.

FIG. 10 shows elution profiles and gel electrophoresis results of a two stage purification process and four column system as shown in FIG. 9. A low expressed His-tagged polypeptide in BEVS media was purified.

FIG. 11A-11B show purification in a four column, NiExcel, desalting, NiNTA, and Superdex® 75, system of a protein expressed in insect cells in baculovirus expression system (BEVS) buffer. FIG. 11A provides a flowchart of the purification process, while FIG. 11B shows the elution profile and an electrophoresis gel of the fractions eluted from the columns.

FIG. 12A-12B show purification in a four column, NiExcel, desalting, NiNTA, and Superdex® 200, system of a protein expressed in CHO cells. FIG. 12A provides a flowchart of the purification process, while FIG. 12B shows the elution profile and an electrophoresis gel of the fractions eluted from the columns.

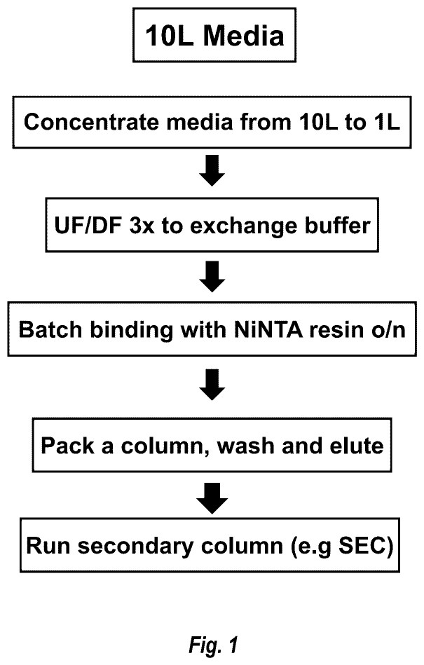

### DETAILED DESCRIPTION OF CERTAIN EMBODIMENTS

### 1. Definitions

Unless otherwise defined, scientific and technical terms used in connection with the present invention shall have the meanings that are commonly understood by those of ordinary skill in the art.

In this application, the use of “or” means “and/or” unless stated otherwise. In the context of a multiple dependent claim, the use of “or” refers back to more than one preceding independent or dependent claim in the alternative only. Also, terms such as “element” or in “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

A “composition” comprising a polypeptide to be purified herein broadly comprises at least one polypeptide of interest and one or more contaminants or impurities, such as other polypeptides and/or other non-polypeptide molecules. In some embodiments, the composition is derived from a cell culture (i.e., from cells used to express the polypeptide), such as a cell lysate or clarified cell lysate, or other type of sample providing a polypeptide expressed in cells and intended for purification. In other cases, the composition may be derived from a polypeptide synthesis reaction. In yet other cases, the composition could be derived from a living organism such as a plant or animal used to express the polypeptide of interest, such as a sample taken from the organism. The composition may be “partially purified” (i.e. having been subjected to one or more prior purification steps such as chromatography) or may be obtained directly from a host cell or organism producing the polypeptide (e.g. the composition a homogenate or harvested cell culture fluid or lysate).

A “contaminant” or “impurity” is a material that is different from the desired polypeptide product. The contaminant may be, without limitation, a variant, fragment, aggregate or derivative of the desired polypeptide (e.g. a variant without a His-tag where the desired polypeptide is His-tagged), another polypeptide, nucleic acid, endotoxins, nucleic acids, lipids and membrane components, cell debris, small molecule chemicals, and other materials that may be found in the preparation of a polypeptide in cells or synthetically.

“Affinity chromatography” or “affinity-based capture” refers to a method of separation based on a specific interaction between molecules of an affinity column and a particular polypeptide to be purified, such as the binding of the polypeptide to a ligand, the binding of a His-tag or other peptide tag to a metal ion or to a specific antibody or the like placed a chromatography matrix.

As used herein, an “immobilized metal affinity chromatography (IMAC)” refers to a type of affinity-based capture where polypeptides are separated from contaminants according to their affinity for metal ions coordinated to ligands on an insoluble matrix. In some embodiments, an IMAC matrix is provided in a column, i.e., an “IMAC column.” In some embodiments, the metal ion is a nickel ion.

The term “matrix” is used herein to refer to a chromatography material, such as an affinity chromatography material. In some embodiments, the matrix may comprise beads or particles comprising a material to which a polypeptide may selectively bind, such as comprising a chelating ligand bound to nickel. In some embodiments, a matrix of affinity chromatography material may be placed in column through which the material to be purified may flow. In other cases, it may be placed in a spin column, or placed on a plate or chip or other device. In some cases, the matrix could comprise beads or particles, such as magnetic particles, that can be separated from a solution, e.g., by introduction of a magnet.

“Size exclusion chromatography (SEC)” is a chromatographic method of separating molecules by size.

An “eluate” as used herein refers to material that has been eluted from a chromatography matrix or column by application of an elution buffer.

The terms “polypeptide” and “protein” are used interchangeably and refer to a polymer of amino acid residues. Such polymers of amino acid residues may contain natural and/or non-natural amino acid residues, and include, but are not limited to, peptides, oligopeptides, dimers, trimers, and multimers of amino acid residues. The terms also include polymers of amino acids that have modifications such as, for example, glycosylation, sialylation, and the like, or that are complexed with other molecules.

In some embodiments, a polypeptide or protein to be purified is an antibody. The term “antibody” herein is used in the broadest sense and encompasses various antibody structures, including but not limited to monoclonal antibodies, polyclonal antibodies, multispecific antibodies (e.g., bispecific antibodies), nanobodies, diabodies, and antigen binding fragments such as Fv, scFv, Fab, (Fab′) 2, and the like, so long as they exhibit antibody-antigen-binding activity.

The term “antigen binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “isolated” or “purified” polypeptide or protein means a polypeptide that has been at least partially separated from one or more contaminants. In some embodiments, a polypeptide is purified to greater than 80%, 90%, 95%, or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of protein and antibody purity, see, e.g., Flatman et al., J. Chromatogr. B 848:79-87 (2007).

As used herein, the term “unpurified polypeptide” or “unpurified protein” with reference to the methods herein is a polypeptide or protein that has not been subjected to any chromatography or other process intended to at least partially separate the polypeptide from contaminants. For example, an unpurified polypeptide may be found in a cell lysate or a solution following peptide synthesis or the like.

The term “buffer exchange” refers to a process of replacing the buffer in which a polypeptide of interest to be purified is found, such as to reduce the concentration of salt in the buffer or to remove components that may interfere with one or more chromatography processes. In some embodiments, a buffer exchange may be performed on a filter or column, such as a “desalting column.”

A “desalting column” is a size exclusion chromatography column having a molecular weight cutoff that is lower than the molecular weight of the polypeptide to be purified so that a higher salt buffer may be removed from the polypeptide to be purified. In some embodiments, such a column may have a molecular weight cut-off of, for example, between 17,000 to 90,000 Da, depending upon the polypeptide to be purified.

As used herein, “equilibration buffer” is an aqueous buffer that is compatible with the polypeptide to be purified and facilitates interaction and binding between the metal ions in a chromatography column, such as an IMAC column and the polypeptide.

An “elution buffer” is used to elute (i.e., remove) a polypeptide that is bound to a column or matrix such as an IMAC or SEC matrix. An elution buffer may elute a polypeptide from a matrix on the basis, for example, of conductivity, pH, charge, ionic strength, or the like.

A “regeneration buffer” may be used to regenerate a chromatography matrix, such as an IMAC matrix, such that it can be re-used.

As used herein, an “automated” or “automatically controlled” process is one that is capable of being run, for example, by a computerized control system with appropriate software, as opposed to a system that requires an active, manual intervention during or between at least one step, such as to move an analyte-containing sample from one part of the system to another. In some embodiments, the process is automated by software that controls the movements or positions of one or more pumps, valves, and/or tees during the course of the process, which movements or positions, in turn, control the flow of buffers and eluates through the system.

### 2. Methods

This disclosure relates, for example, to methods and systems for purifying a polypeptide. As described in more detail in the Examples below, His tags and His tag affinity resins are often used to generate very pure protein reagents. However, His tagged secreted proteins in cell culture media however cannot be loaded directly over many nickel-containing IMAC matrices because the media chelates the nickel off the resin, limiting options for how such proteins can be purified. A traditional, low throughput, way to solve this problem is to do buffer exchange prior to loading the conditioned media over the IMAC matrix. Buffer exchanging each expression is a time consuming, very hands on process that makes the purification of secreted His-tagged proteins low throughput. Certain IMAC matrices such as Ni-excel are not striped by conditioned media making them amenable for directly loading without the need to buffer exchange. However, we observed that such matrices bound nonspecifically to contaminating proteins in the cell media compositions, which impurities could not be sufficiently removed by further purification steps. For example, loading mammalian or insect cell culture media, such as Baculovirus (BEVS) and CHO media, comprising a His-tagged protein at a low expression level directly over Ni-Excel, even with a further size exclusion (SEC) step, resulted in material with very low purity. For polypeptides expressed in CHO media, both high and low expressors showed significant unspecific binding that could not be removed entirely by SEC. We tested whether adding a low concentration of imidazole to the conditioned media prior to loading and observed that this did not result in an increase in purity, and also resulted in a significant loss in protein yield.

The methods disclosed herein solve these myriad problems in purifying His-tagged polypeptides from cell culture media such as mammalian and insect cell culture media, such as BEVS and CHO media. In some embodiments, the methods include (a) loading a composition comprising the polypeptide onto a first immobilized metal affinity chromatography (IMAC) matrix; (b) eluting the polypeptide from the first IMAC matrix with an elution buffer to form a first eluate comprising the polypeptide; (c) conducting a buffer exchange to remove the elution buffer from the first eluate; (d) loading the first eluate from step (c) onto a second IMAC matrix; and (e) eluting the polypeptide from the second IMAC matrix with a second elution buffer to form a second eluate comprising the polypeptide.

In some embodiments, the composition is derived from cell culture, i.e., of host cells that express the polypeptide to be purified. Thus, the composition from which the polypeptide is to be purified may be a cell lysate or a clarified cell lysate or the like. Hence, in some embodiments the composition comprises cell culture media. In some embodiments, the cell culture media can be artificial media, serum containing media, serum-free media, chemically defined media, or protein-free media. Examples of cell culture media include, but are not limited to media used with mammalian and insect cells. Examples of such media include Baculovirus Expression Vector Systems (BEVS) media, Chinese Hamster Ovary (CHO) media, N293 cell media, RPMI 1640 media, and Dulbecco's Modified Eagle Medium (DMEM). In some embodiments, the cell culture media can be conditioned cell culture media. Conditioned cell culture media includes proteins and/or cytokines secreted by cells. In some embodiments, the composition may also include non-protein contaminants such as nucleic acids, lipids, and the like. In other embodiments, the composition may be from a protein synthesis chemical reaction, and may contain contaminants such as non-fully reacted polypeptide components and the like.

In some embodiments, the polypeptide comprises a polyhistidine tag (His-Tag) or other suitable tag that is intended to specifically bind to an IMAC matrix, such as a matrix comprising a chelating ligand bound to nickel. The His-tagged polypeptide can be produced any suitable cell or could be added during peptide synthesis reactions. Examples of cells that can express His-tagged polypeptides, include but are not limited to, bacteria, insect cells, E. coli, or mammalian cells. In some embodiments, the His-tagged polypeptide is produced in insect cells using BEVS media or in CHO cells using CHO media. CHO media generally comprises Dulbecco's Modified Eagle Medium (DMEM) supplemented with fetal bovine serum or an equivalent serum, and may further comprise L-glutamine as well as an antibiotic such as penicillin and streptomycin to prevent bacterial growth. In some cases, CHO media comprises DMEM, 10% fetal bovine serum (FBS), 2 mM L-glutamine, 100 U/mL penicillin and 100 μg/mL streptomycin. BEVS media generally comprises RPMI 1640 medium supplemented with FBS or equivalent serum, for example, in some cases with 10% FBS, and may further contain a surfactant, such as Pluronic® F68, L-glutamine, penicillin, and streptomycin. In some cases, BEVS media also comprises 100 U/mL penicillin and 100 μg/mL streptomycin. Media such as BEVS and CHO media also comprise essential amino acids and buffers to closely mimic the composition of mammalian cells.

The polypeptide to be purified may be any His-tagged polypeptide that is capable of binding to IMAC matrices used herein. Examples of polypeptides include, but are not limited to, antibodies, cell receptors, intracellular proteins, secreted proteins, membrane proteins, and others. In some cases, the polypeptide may be an antigen or target for antibody binding. In some embodiments, the polypeptide is secreted from cells of the cell culture, i.e., is a secreted polypeptide.

The loading of the composition containing the polypeptide on the first IMAC matrix can be conducted by any known means available to one of ordinary skill in the art. For example, the composition can be loaded in an automated fashion where a pump draws the composition from a reservoir containing the composition and pumps the fluid onto a column containing first IMAC matrix. In some embodiments, the composition can be loaded into a tube (i.e. loop) and a pump directs buffer from a reservoir through the tube containing the composition and onto a column containing the first IMAC matrix. Alternatively, the composition could be loaded manually (i.e. pouring) the composition into the first IMAC matrix, or by other methods that do not utilize a pump, or generally by gravity flow.

In some embodiments, the first IMAC matrix comprises a chelating ligand that is bound to nickel ions. Polypeptides comprising a His-tag or similar tag that binds to a nickel-containing matrix are brought into contact with the first IMAC matrix are allowed to bind to the matrix until they are eluted using an elution buffer. In some embodiments, the first IMAC matrix comprises agarose beads and a chelating ligand bound to nickel. In some embodiments, the chelating ligand on the first IMAC matrix is an oxidation-tolerant proteinaceous ligand that binds polyhistidine. In some embodiments, the chelating ligand comprises sulfopropyl, sulfoethyl, carboxymethyl, trimethylammonium, or diethylaminoethyl groups.

In some embodiments, the first IMAC matrix retains nickel when exposed to BEVS or CHO media. In some instances, cell culture media can contain a stripping agent that, when exposed to an IMAC matrix comprising nickel, will result in nickel being washed or leached from the matrix. In some embodiments, the first IMAC matrix is any matrix described in EP 1276716, which is incorporated by reference for all it contains. In some embodiments, the first IMAC matrix is a matrix sold under the brand name Ni Sepharose™ Excel or HisTrap™ Excel (Sigma-Aldrich Co.) (i.e., Ni Excel herein) or an equivalent of such a matrix.

In some embodiments, the first IMAC matrix is contained within a column. In some embodiments, the first IMAC column comprises a bed volume of 2 to 20 ml. In some embodiments, the first IMAC column comprises a bed volume of 2, 3, 4, 5, 6, 7, 8, 9, 10, 11, 12, 13, 14, 15, 16, 17, 18, 19, or 20 ml. In other embodiments, the first IMAC matrix is contained within a spin column, and may comprise a bed volume of, for example, 0.5, 0.75, 1, or 2 ml.

In some embodiments, the first IMAC matrix can be washed with a wash buffer after the polypeptide has been loaded onto the first IMAC matrix. In some embodiments, the wash buffer comprises imidazole. The amount of imidazole in the wash buffer can range from 0 to 20 mM. In some embodiments, the amount of imidazole in the wash buffer can range from 5 to 15 mM. In some embodiments, the amount of imidazole in the wash buffer can be 5, 6, 7, 8, 9, 10, 11, 12, 13, 14, or 15 mM. In some embodiments, the wash buffer comprises trisodium phosphate (Na₃PO₄). The amount of Na₃PO₄ in the wash buffer can range from 20 to 70 mM. In some embodiments, the amount of Na₃PO₄ in the wash buffer can range from 40 to 60 mM. In some embodiments, the amount of Na₃PO₄ in the wash buffer can be 45, 50, or 55 mM. In some embodiments, the wash buffer comprises sodium chloride (NaCl). The amount of NaCl in the wash buffer can range from 5 to 35 mM. In some embodiments, the amount of NaCl in the wash buffer can range from 15 to 25 mM. In some embodiments, the wash buffer can include a surfactant. In some embodiments, the amount of NaCl in the wash buffer can be 15, 20, or 25 mM. In some embodiments, the wash buffer comprises 50 mM Na₃PO₄, 20 mM NaCl, and 10 mM imidazole. In some embodiments, the wash buffer comprises 50 mM Na₃PO₄, 20 mM NaCl, 10 mM imidazole, and 0.1% Triton X-114. In some embodiments, the amount of wash buffer used to wash the first IMAC column can range from one to five times the bed volume of the column. In some embodiments, the IMAC matrix can be first washed with buffer without surfactant and then washed with buffer containing a surfactant.

The polypeptide can be eluted from the first IMAC matrix using an elution buffer. In some embodiments, the elution buffer comprises imidazole. The amount of imidazole in the elution buffer can range from 0 to 1000 mM. In some embodiments, the amount of imidazole in the elution buffer can range from 100 to 1000 mM, 100 to 800 mM, 200 to 600 mM, or 300 to 500 mM. In some embodiments, the amount of imidazole in the elution buffer can be 100, 150, 200, 250, 300, 350, 400, 450, 500, or 550 mM. In some embodiments, the elution buffer comprises trisodium phosphate (Na₃PO₄). The amount of Na₃PO₄ in the elution buffer can range from 20 to 70 mM. In some embodiments, the amount of Na₃PO₄ in the elution buffer can range from 40 to 60 mM. In some embodiments, the amount of Na₃PO₄ in the elution buffer can be 45, 50, or 55 mM. In some embodiments, the elution buffer comprises sodium chloride (NaCl). The amount of NaCl in the elution buffer can range from 5 to 35 mM. In some embodiments, the amount of NaCl in the elution buffer can range from 15 to 25 mM. In some embodiments, the amount of NaCl in the elution buffer can be 15, 20, or 25 mM. In some embodiments, the elution buffer comprises 50 mM Na₃PO₄, 20 mM NaCl, and 400 mM imidazole. In some embodiments, the amount of elution buffer used to elute the polypeptide from the first IMAC column can range from one to five times the bed volume of the column.

In some embodiments, the method includes conducting a buffer exchange to separate the elution buffer in the first eluate from the polypeptide of interest. In this step, for example, if imidazole is utilized in the elution buffer, or a high salt concentration is used in the elution buffer, such imidazole or salt can be is substantially removed in preparation for loading the first eluate onto the second IMAC matrix. In some embodiments, the buffer exchange comprises passing the first eluate over a desalting column. The polypeptide passes through the desalting column more quickly than lower molecular weight molecules, thus allowing for collection of polypeptide while retaining lower molecular weight species such as imidazole. In some embodiments, the desalting column comprises a bed volume of 2 to 20 ml. In some embodiments, the desalting column comprises a bed volume of 2, 3, 4, 5, 6, 7, 8, 9, 10, 11, 12, 13, 14, 15, 16, 17, 18, 19, or 20 ml. Other means for conducting the buffer exchange include using dialysis or ultrafiltration, using a membrane or filter that traps polypeptides but allows buffer components and small molecules to pass through.

After the buffer exchange step, the first eluate is loaded on the second IMAC matrix. The loading of the first eluate on the second IMAC matrix can be conducted similarly to how the composition was loaded onto the first IMAC matrix. In some embodiments, the eluate from the desalting column is collected in a tube (i.e. loop) that is in-line with the second IMAC matrix. Once the eluate is collected in the tube, the pump can direct the fluid onto the second IMAC matrix.

In some embodiments, the second IMAC matrix comprises nickel. In some embodiments, the second IMAC matrix comprises agarose beads and a nitrilotriacetic acid chelation moiety bound to nickel (Ni NTA). The nitrilotriacetic acid (NTA) chelation moiety is attached to the agarose beads. In other embodiments, the first and second IMAC are the same. Any His-tagged polypeptide that is capable of binding to a nickel chelate in the first eluate may bind to the nickel on the second IMAC matrix. In some embodiments, the second IMAC matrix is contained within a column. In some embodiments, the second IMAC column comprises a bed volume of 2 to 20 ml. In some embodiments, the second IMAC column comprises a bed volume of 2, 3, 4, 5, 6, 7, 8, 9, 10, 11, 12, 13, 14, 15, 16, 17, 18, 19, or 20 ml. In other embodiments, the second IMAC matrix is contained within a spin column, and may comprise a bed volume of, for example, 0.5, 0.75, 1, or 2 ml.

In some embodiments, the second IMAC matrix can be washed with a wash buffer after the polypeptide has been loaded onto the second IMAC matrix. In some embodiments, the wash buffer comprises imidazole. The amount of imidazole in the wash buffer can range from 0 to 20 mM. In some embodiments, the amount of imidazole in the wash buffer can range from 5 to 15 mM. In some embodiments, the amount of imidazole in the wash buffer can be 5, 6, 7, 8, 9, 10, 11, 12, 13, 14, or 15 mM. In some embodiments, the wash buffer comprises trisodium phosphate (Na₃PO₄). The amount of Na₃PO₄ in the wash buffer can range from 20 to 70 mM. In some embodiments, the amount of Na₃PO₄ in the wash buffer can range from 40 to 60 mM. In some embodiments, the amount of Na₃PO₄ in the wash buffer can be 45, 50, or 55 mM. In some embodiments, the wash buffer comprises sodium chloride (NaCl). The amount of NaCl in the wash buffer can range from 5 to 35 mM. In some embodiments, the amount of NaCl in the wash buffer can range from 15 to 25 mM. In some embodiments, the amount of NaCl in the wash buffer can be 15, 20, or 25 mM. In some embodiments, the wash buffer comprises 50 mM Na₃PO₄, 20 mM NaCl, 10 mM imidazole, and 0.1% Triton X-114. In some embodiments, the wash buffer can include a surfactant. In some embodiments, the wash buffer comprises 50 mM Na₃PO₄, 20 mM NaCl, and 10 mM imidazole. In some embodiments, the amount of wash buffer used to wash the second IMAC column can range from one to five times the bed volume of the column. In some embodiments, the IMAC matrix can be first washed with buffer without surfactant and then washed with buffer containing a surfactant.

The polypeptide can be eluted from the second IMAC matrix using an elution buffer. In some embodiments, the elution buffer comprises imidazole. The amount of imidazole in the elution buffer can range from 0 to 1000 mM. In some embodiments, the amount of imidazole in the elution buffer can range from 100 to 1000 mM, 100 to 800 mM, 200 to 600 mM, or 300 to 500 mM. In some embodiments, the amount of imidazole in the elution buffer can be 100, 150, 200, 250, 300, 350, 400, 450, 500, or 550 mM. In some embodiments, the elution buffer comprises trisodium phosphate (Na₃PO₄). The amount of Na₃PO₄ in the elution buffer can range from 20 to 70 mM. In some embodiments, the amount of Na₃PO₄ in the elution buffer can range from 40 to 60 mM. In some embodiments, the amount of Na₃PO₄ in the elution buffer can be 45, 50, or 55 mM. In some embodiments, the elution buffer comprises sodium chloride (NaCl). The amount of NaCl in the elution buffer can range from 5 to 35 mM. In some embodiments, the amount of NaCl in the elution buffer can range from 15 to 25 mM. In some embodiments, the amount of NaCl in the elution buffer can be 15, 20, or 25 mM. In some embodiments, the elution buffer comprises 50 mM Na₃PO₄, 20 mM NaCl, and 400 mM imidazole. In some embodiments, the amount of elution buffer used to elute the polypeptide from the second IMAC column can range from one to five times the bed volume of the column. In some embodiments, the elution buffer used for the first IMAC matrix and the second IMAC matrix are the same.

In some embodiments, the time that the polypeptide elutes from the first and second IMAC columns can be determined experimentally using UV absorbance of eluate fractions, such as at a wavelength suitable for observing polypeptides, e.g. 280 nm or the like. The elution time window can be determined and the correct fractions can be collected. For example, during a particular elution time window, eluate can be collected in a tube (i.e. loop) in preparation for processing through the next column. Outside the elution time window, eluate can be directed to waste collector. In some embodiments, an eluate may be further analyzed by, for example, gel electrophoresis or mass spectrometry or other detection techniques in order to assess its purity and yield.

If desired, the second eluate can be further purified using size exclusion chromatography (SEC) or other chromatography methods such as anion or cation exchange, for example. In some embodiments, the method includes a step comprising conducting SEC on the second eluate. The step of conducting SEC can include loading the second eluate onto an SEC column. The particular pore size of the solid phase in the SEC column can be selected based on the molecular weight of the polypeptide. For example, in some embodiments a Superdex® 75 or a Superdex® 200, or equivalents, may be used as an SEC matrix. In some embodiments, eluate from an SEC matrix may be collected in different fractions for later analysis, such as on a 96 well plate.

In some embodiments, the method includes detecting the purified polypeptide collected from any of the first or second IMAC eluates, or, if SEC is conducted, from the SEC eluate. Various methods can be used to detect elution of the polypeptide from the SEC column. In some embodiments, UV spectroscopy can be used to detect elution of the polypeptide from the SEC column. Other methods include, but are not limited to, electrophoresis or mass spectrometry.

In some embodiments, the method includes regenerating the first IMAC matrix and/or the second IMAC matrix, and if used, further chromatography matrix such as SEC, by washing with a regeneration buffer. In some embodiments, the regeneration buffer comprises sodium hydroxide. In some embodiments, the regeneration buffer comprises 0.1 to 1 M sodium hydroxide. In some embodiments, the regeneration buffer comprises 0.1, 0.2, 0.3, 0.4, 0.5, 0.6, 0.7, 0.8, 0.9, or 1 M sodium hydroxide. In some embodiments, an equilibration buffer is then run over the matrix to remove such sodium hydroxide and prepare the matrix for a new polypeptide purification process.

In some embodiments, a method for purifying a His-tagged polypeptide herein includes (a) loading a composition comprising the His-tagged polypeptide onto a first immobilized metal affinity chromatography (IMAC) matrix; (b) eluting the His-tagged polypeptide from the first IMAC matrix with an elution buffer to form a first eluate comprising the polypeptide; (c) conducting a buffer exchange to remove the elution buffer from the first eluate using a desalting column; (d) loading the first eluate from step (c) onto a second IMAC matrix, wherein the second IMAC matrix comprises agarose beads and a nitrilotriacetic acid chelation moiety bound to nickel; and (e) eluting the His-tagged polypeptide from the second IMAC matrix with a second elution buffer to form a second eluate comprising the His-tagged polypeptide. In some such embodiments, the first IMAC matrix comprises a chelating ligand that is bound to nickel ions. Polypeptides comprising a His-tag or similar tag that binds to a nickel-containing matrix are brought into contact with the first IMAC matrix are allowed to bind to the matrix until they are eluted using an elution buffer. In some embodiments, the first IMAC matrix comprises agarose beads and a chelating ligand bound to nickel. In some embodiments, the chelating ligand on the first IMAC matrix is an oxidation-tolerant proteinaceous ligand that binds polyhistidine. In some embodiments, the chelating ligand comprises sulfopropyl, sulfoethyl, carboxymethyl, trimethylammonium, or diethylaminoethyl groups. In some embodiments, the first IMAC matrix retains nickel when exposed to BEVS or CHO media. For example, in some instances, cell culture media can contain a stripping agent that, when exposed to an IMAC matrix comprising nickel, will result in nickel being washed or leached from the matrix. In some embodiments, the first IMAC matrix is any matrix described in EP 1276716, which is incorporated by reference for all it contains. In some embodiments, the first IMAC matrix is a matrix sold under the brand name Ni Sepharose™ Excel or HisTrap™ Excel (Sigma-Aldrich Co.) (i.e., Ni Excel herein) or an equivalent of such a matrix.

In some embodiments, the first and second IMAC matrices are contained within a column. In some embodiments, the first and second IMAC matrices are columns comprising a bed volume of 2 to 20 ml. In some embodiments, the first and second IMAC columns comprise a bed volume of 2, 3, 4, 5, 6, 7, 8, 9, 10, 11, 12, 13, 14, 15, 16, 17, 18, 19, or 20 ml. Such a process may further include an SEC step following chromatography with the two IMAC matrices.

In some embodiments, where IMAC and/or SEC matrices are provided on columns, a controller directs flow through columns that contain the first IMAC matrix and the second IMAC matrix the method is automated. In some embodiments, a controller directs flow through columns that contain the first IMAC matrix, the desalting column, the second IMAC matrix, and the SEC column, and the method is automated.

Flow rates for the composition, buffers, eluates through the columns can be selected based on the size of the columns. The flow rate through the columns (IMAC, desalting, and SEC) can be the same or different. In some embodiments, the flow rate through the first and second IMAC columns can be 2 to 8 ml/min. In some embodiments, the flow rate through the first and second IMAC columns can be 5 ml/min. In some embodiments, the flow rate through the desalting column can be 5 to 15 ml/min. In some embodiments, the flow rate through the first and second IMAC columns can be 10 ml/min.

In some embodiments, the methods herein are advantageous over prior methods using a single IMAC column in that there is no need to first exchange the buffer of a starting composition that comprises cell culture media, such as BEVS or CHO media before the composition is loaded onto an IMAC matrix, such as a column. Such a buffer exchange process to remove cell culture media can be relatively time-consuming. At the same time, the processes herein allow for greater purification of a His-tagged polypeptide than in a traditional one-IMAC method, as can be seen in the Examples and figures of the disclosure. Methods herein are also compatible with both high and low expressing polypeptides from particular cell culture host cells. Methods herein may also be partially or fully automated. Automated methods in particular may also allow for high-throughput protein preparation that is not otherwise possible if a starting composition such as a cell lysate must be loaded onto an IMAC matrix in batches or if a cell lysate must first be buffer exchanged before chromatography to be compatible with an IMAC composition. Further, as described in more detail in the following section, systems may be designed to carry out methods herein such that several large scale polypeptide expressions may be purified in tandem with minimal hands-on requirements.

### 3. Systems

The present disclosure also relates to systems capable of conducting methods described herein, including those described in the section above. In some cases, the system comprises (a) an injection valve for injecting a composition containing the polypeptide into the system, wherein the injection valve is connected to a first immobilized metal affinity chromatography (IMAC) matrix and a pump to control flow of the sample through the first IMAC matrix; (b) a column valve for controlling flow path of an equilibration buffer, an elution buffer, and an SEC buffer; (c) a desalting column or similar device used for buffer exchange, which is configured to receive flowthrough or eluate from the first IMAC matrix; and (d) a second IMAC matrix configured to receive flowthrough or eluate from the desalting column; and (f) a detection device, such as a UV spectrometer or mass spectrometer, for detecting the polypeptide in the system. In some cases, the system further comprises an additional chromatography column for further purification of the polypeptide, such as (e) a size exclusion chromatography (SEC) column configured to receive flowthrough or eluate from the second IMAC matrix. In some cases, the injection valve and the column valve can be automatically controlled.

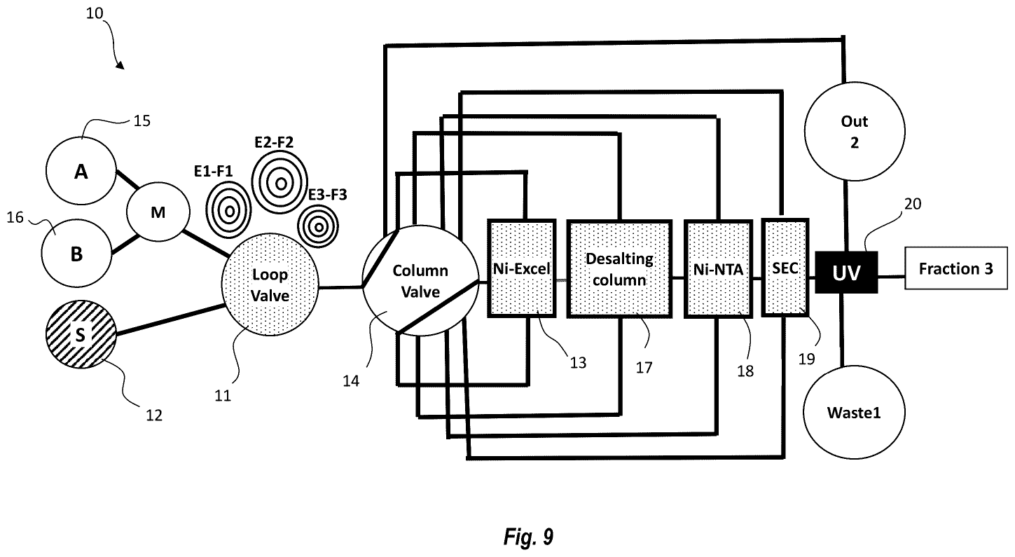

For instance, FIG. 9 shows an embodiment of an exemplary system 10 for purifying a polypeptide according to methods herein. For example, the system 10 includes four separate columns, two IMAC columns (a) an injection valve 11 for injecting a composition 12 containing the polypeptide into the system 10, wherein the injection valve 11 is connected to a first immobilized metal affinity chromatography (IMAC) column 13 and a pump to control flow of the sample through the first IMAC column 13; (b) a column valve 14 for controlling flow path of an equilibration buffer 15, an elution buffer 16, and an SEC buffer; (c) a desalting column 17 configured to receive flowthrough or eluate from the first IMAC column 13; and (d) a second IMAC column 18 configured to receive flowthrough or eluate from the desalting column 17; (e) a size exclusion chromatography (SEC) column 19 configured to receive flowthrough or eluate from the second IMAC column 18; and (f) a detection device 20 for detecting the polypeptide in the system. Optionally, the system may also comprise a mixer (M in FIG. 9) that mixes mobile phase buffer gradients, if such gradients are utilized in the chromatography methods.

In some embodiments, the system incorporates the controller, pumps, and interface of a commercial multi-column chromatography system, such as an AKTA chromatography system (Cytiva). Each column can be connected, in series, to tubing of such a chromatography system. In some embodiments, the injection valve and the column valve can be automatically controlled to direct buffers through the appropriate columns at the appropriate times.

As described above, systems disclosed herein may have certain advantages over systems that utilize only one IMAC matrix. For example, an entire starting composition may be loaded onto the IMAC matrices without being broken down into batches. In some embodiments, there is no need to first exchange the buffer of a starting composition that comprises cell culture media, such as BEVS or CHO media before the composition is loaded onto an IMAC matrix, while at the same time, allowing for greater purification of a His-tagged polypeptide than in a traditional one-IMAC method, as can be seen in the Examples and figures of the disclosure. Systems herein may be used with both high and low expressing polypeptides from particular cell culture host cells. Automated systems in particular may also allow for high-throughput protein preparation that is not otherwise possible if a starting composition such as a cell lysate must be loaded onto an IMAC column or other matrix in batches or if a cell lysate must first be buffer exchanged before chromatography to be compatible with an IMAC composition. Further, systems such as shown in FIG. 9 may carry out methods herein such that several large scale polypeptide expressions may be purified in tandem with minimal hands-on requirements. For example, a 5 mL Ni Excel column used in methods and systems herein has been used in about 30 1 L runs with compositions in CHO or BEVS media over a 3 month period with no change of color or loss of capacity.

### EXAMPLES

### Example 1. Imidazole Effect on Protein Yield from Ni Excel

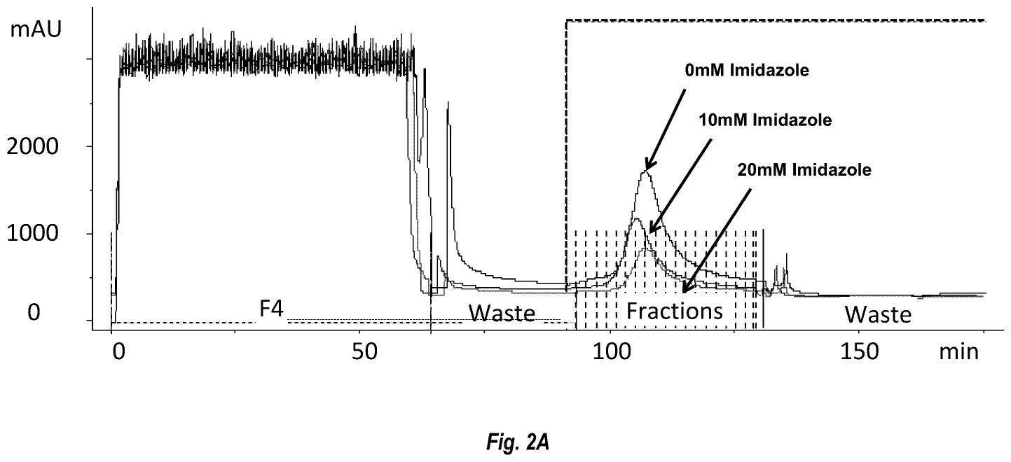

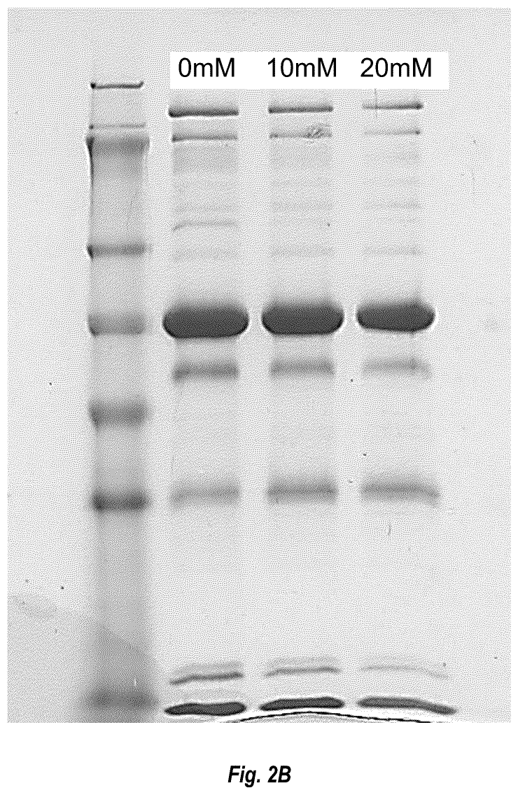

The effect of adding up to 20 mM imidazole on protein purity and yield from a Ni Excel column (Cytiva Cat #17371201) was tested by adding a composition of a polypeptide to be purified in CHO media onto the column with 0, 10, or 20 mM imidazole. Generally, imidazole may be added when performing metal ion affinity chromatography to purify a particular polypeptide, as it can assist in removing impurities due to nonspecific binding to the chromatography matrix. Filtered media (over a 0.2 micron filter) were directly loaded on the column; no additives were added. The column was pre-equilibrated with 50 mM of sodium phosphate, 200 mM of NaCl, and 10 mM imidazole at a pH of 8, 2 mM NaN₃. This same buffer was then used to wash the column. A second wash was performed with 50 mM Tris Na₃PO₄, 200 mM NaCl, 10 mM Imidazole, 2 mM NaN₃, 0.1% Triton-X (TX) 114. The bound polypeptide was eluted in a buffer comprising 50 mM Tris Na₃PO₄, 200 mM NaCl, 400 mM Imidazole, and 2 mM NaN₃. Results in FIG. 2A and FIG. 2B show that increased imidazole concentration resulted in a loss in protein yield, as depicted by the reduced size of the elution traces shown in FIG. 2A, without a significant improvement in purity, as indicated in FIG. 2B, which provides a gel electrophoresis of the collected fractions shown in FIG. 2A.

The effect of washing a Ni Excel column (Cytiva) with different concentrations of imidazole on protein purity and yield was further tested with a 1 L starting composition of a high expressor, secreted His-tagged protein produced in Baculovirus (BEVS) media and loaded onto a 5 mL Ni Excel column in a buffer containing no imidazole. Wash buffers containing 10, 20, 30, 40, or 50 mM imidazole were tested. Each wash buffer had a pH of 8 and contained 50 mM of sodium phosphate and 200 mM of NaCl. The His-tagged protein was eluted from the Ni Excel column using an elution buffer containing 50 mM of sodium phosphate, 200 mM of NaCl, and 300-400 mM imidazole at a pH of 8. The Ni Excel column was regenerated using 0.5 M NaOH. A buffer comprising 50 mM sodium phosphate, pH 8, 200 mM NaCl, and 10 mM imidazole was chosen as a wash buffer.

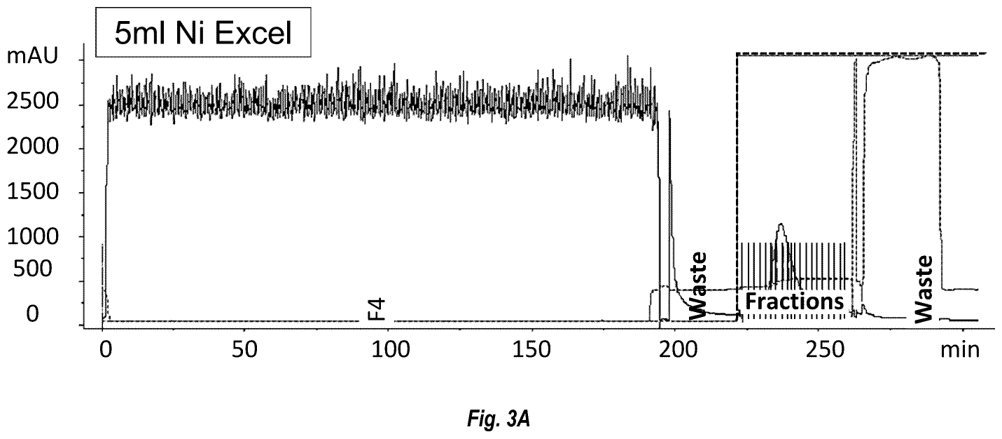

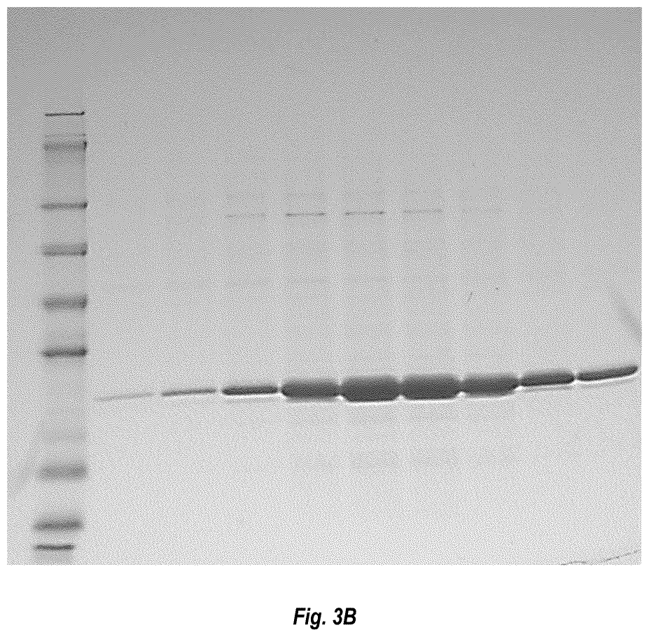

FIG. 3A shows a UV trace of solution exiting the Ni Excel column. The peak at about 240 min corresponds with the eluted protein from the Ni Excel column. No loss in yield was observed up to concentration of 50 mM imidazole in the wash buffer. FIG. 3B shows elution profiles of fractions of the eluted His-tagged polypeptide corresponding to minutes about 220 to about 260 as shown in the trace of FIG. 3A. Elution was tested with both 300 and 400 mM imidazole in the elution buffer. A buffer containing 50 mM sodium phosphate, pH 8, 200 mM NaCl, and 400 mM imidazole gave a sharper peak in the gel and was chosen as an elution buffer.

### Example 2. Two Step Automated Purification (BEVS Media) Using Ni Excel and SEC

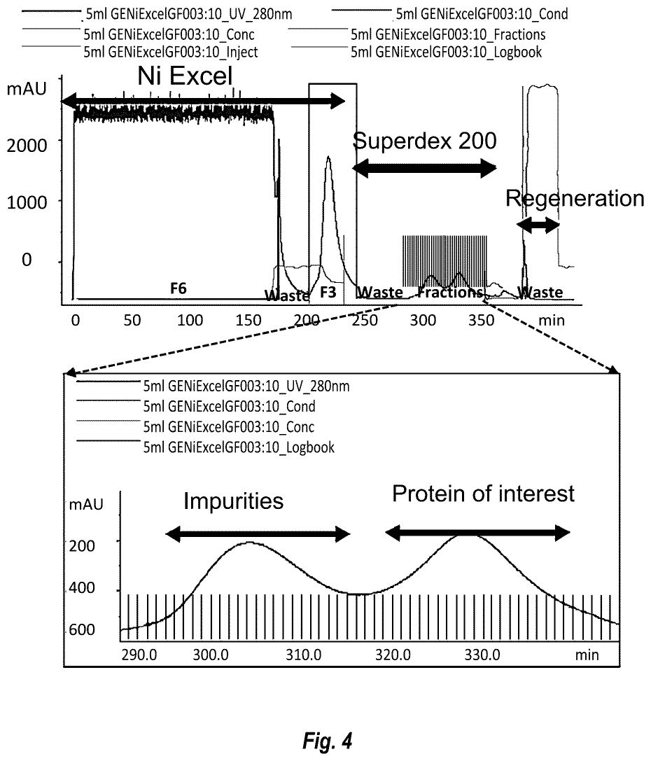

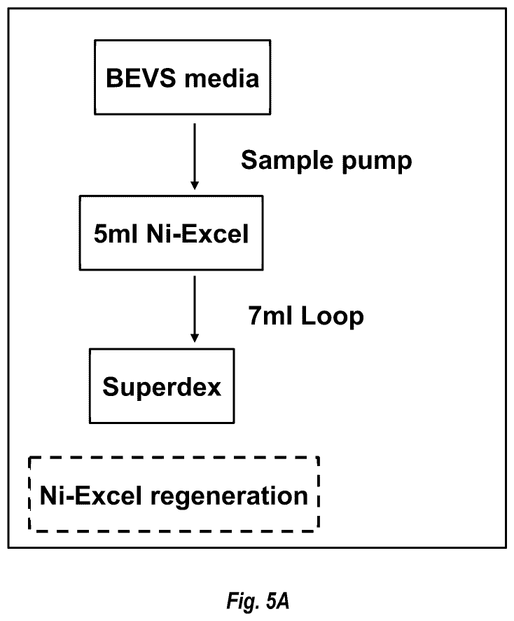

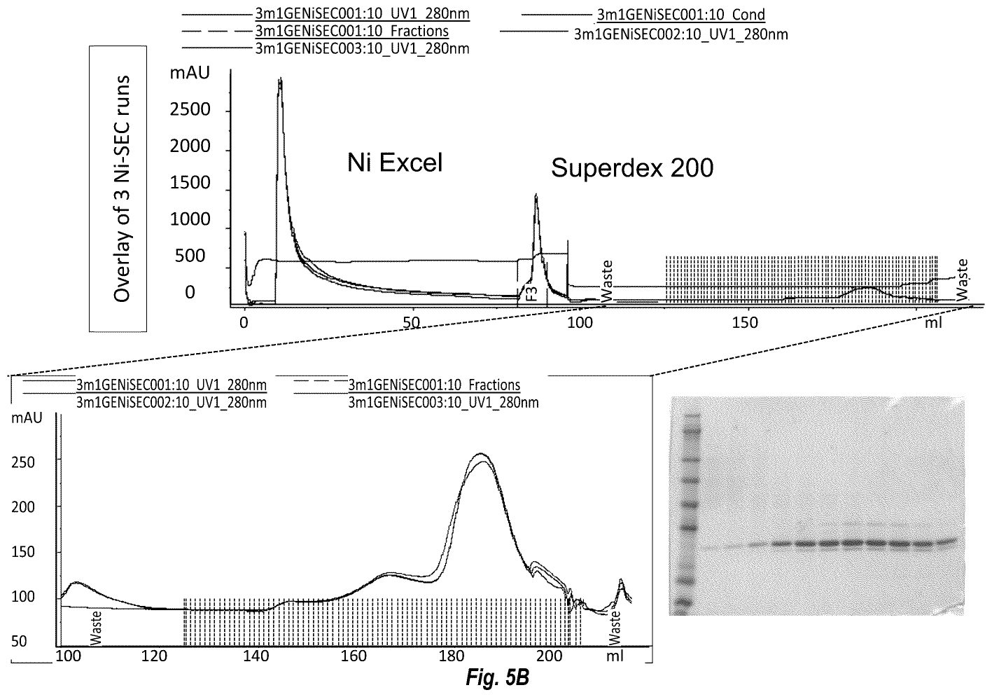

A two step automated purification system was tested that included a Ni Excel column (Cytiva, Cat #17371201) and a Superdex 200 (SEC) column. FIG. 5A shows a flow chart of the steps and some features of the two step procedure. A high expressor, secreted His-tagged protein was produced in BEVS media and loaded at a 1 L volume onto a 5 mL Ni Excel column. The wash buffer contained 50 mM of sodium phosphate, 200 mM of NaCl, and 10 mM imidazole at a pH of 8 and the elution buffer contained 50 mM of sodium phosphate, 200 mM of NaCl, and 400 mM imidazole at a pH of 8. FIG. 4 shows separation of the His-tagged protein of interest from the earlier eluting impurities. The total run time for the nickel and SEC column purification was 7 hours with minimal hands-on time required.

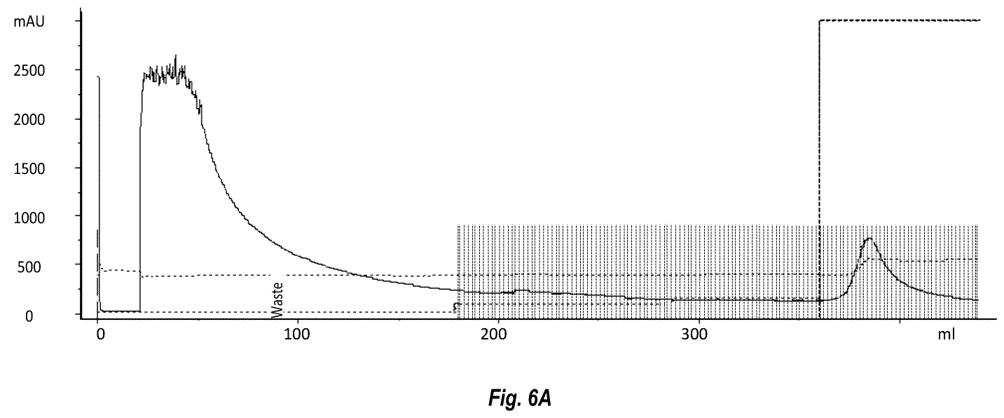

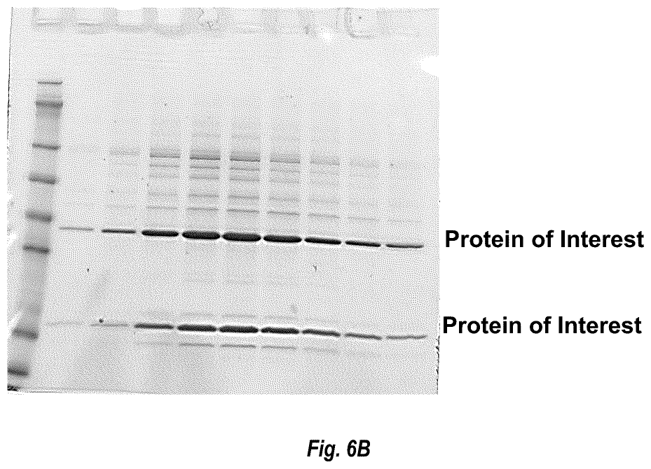

For BEVS media, when the level of expression of the His-tagged protein is high the protein eluted out of the column was pure enough that a size exclusion (SEC) step rendered it over 95% pure (see FIG. 5B). This purification process was also performed with a low expressor, secreted protein in BEVS media, as shown in FIGS. 6A-6B. As shown in those figures, this polypeptide could not be purified to a high enough purity using out of a Ni Excel column that an SEC will suffice to remove the remaining purities (see FIGS. 6A and 6B). This may be because Ni Excel shows more unspecific binding to non-His-tagged proteins than other IMAC matrices such as Ni NTA.

### Example 3. Two Step Automated Purification (CHO Media)

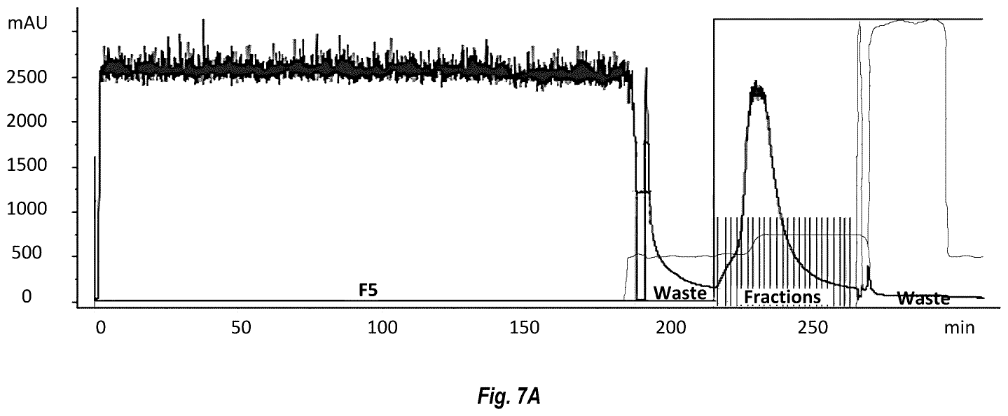

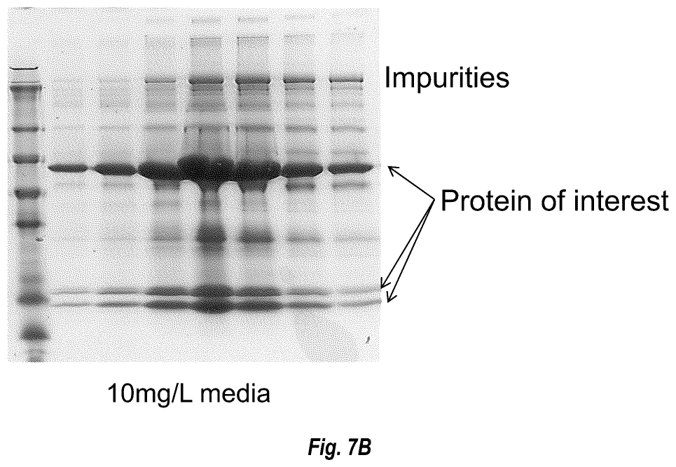

Direct loading of His-tagged protein in CHO media was also tested in this Example. Specifically, filtered media (filtered over a 0.2 micron filter) containing a His-tagged heterodimeric polypeptide to be purified was loaded directly onto a Ni Excel column (Cytiva Cat #17371201) that had been pre-equilibrated in a buffer comprising 40 mM sodium phosphate, 200 mM NaCl, and 10 mM imidazole at pH 8, with 2 mM sodium azide. This same buffer was used for a first wash of the column, followed by a second wash in 50 mM Tris sodium phosphate, 200 mM NaCl, 10 mM imidazole, 2 mM sodium azide, and 0.1% TX 114. The polypeptide was eluted in 50 mM Tris sodium phosphate, 200 mM NaCl, 400 mM imidazole, and 2 mM sodium azide. FIG. 7A shows the UV trace at 280 nm of solution eluted from the Ni Excel column and FIG. 7B shows the protein content in the fractions collected from the Ni Excel column analyzed by gel electrophoresis. As can be seen in FIG. 7B, a considerable amount of impurities were present in the fractions containing the protein of interest.

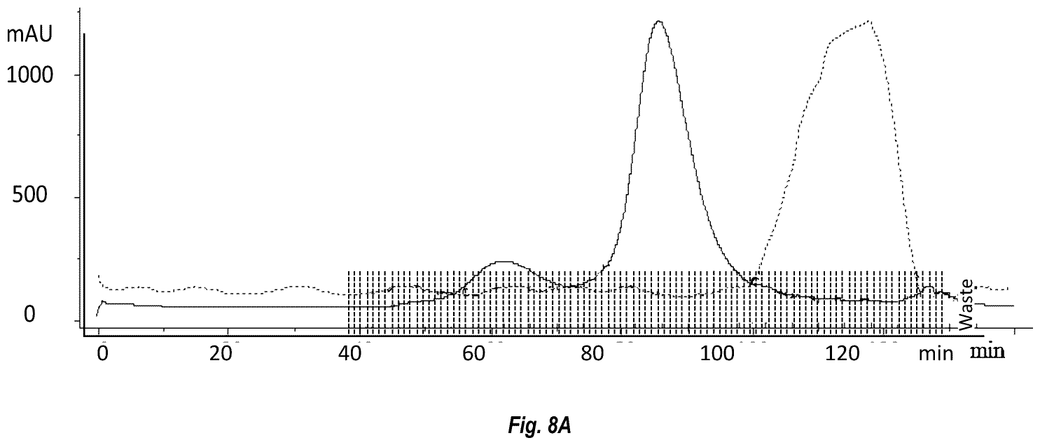

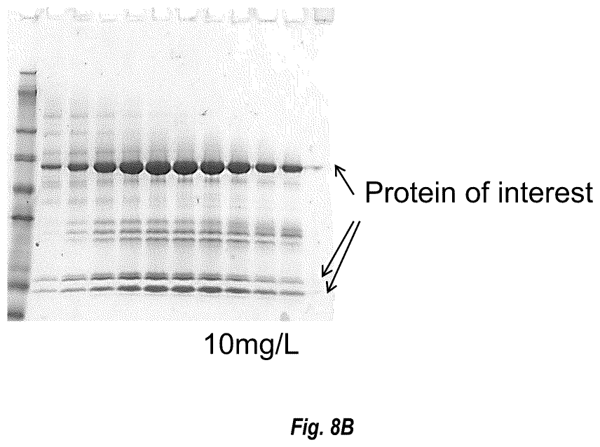

Further purification by SEC chromatography was then conducted. However, the SEC procedure was unable to fully remove impurities from the His-tagged polypeptide, as shown in FIG. 8A and FIG. 8B. Specifically, FIG. 8A shows a UV trace at 280 nm of eluate from the SEC column, with each peak representing each subunit of the heterodimeric polypeptide, and FIG. 8B shows an electrophoresis run of the eluted polypeptide species with arrows denoting the polypeptide subunits of interest. Additionally, the white box in FIG. 7A at about 180 minutes depicts impurities that were removable by the subsequent SEC chromatography step, while FIG. 8B shows that other impurities remained.

Thus, in CHO media, both high and low expressors show a lot of unspecific binding that cannot be removed entirely by SEC and require an additional purification step. Adding 10 or 20 mM imidazole to the conditioned media prior to loading was tested; however, this did not result in an increase in purity of the Ni-eluate but resulted in a significant loss in protein yield.

### Example 4. Four Column Purification Using Ni Excel, Ni NTA, and SEC

A system using two nickel columns in tandem was next tested to determine whether such a set-up would improve purity for high and low expressors from BEVS and CHO media. A four column automated purification system was tested that included a 5 mL Ni Excel column (Cytiva, Cat #17371201), desalting column, 5 mL Ni NTA column (Qiagen, Cat #30761), and a Superdex 200 (SEC) column. The columns were connected to a UNICORN 7.3.0 AKTA purification system. The conditioned media was filtered over a 0.2 micron filter and loaded onto a 5 ml Ni Excel resin pre-equilibrated with 50 mM Na₃PO₄, 20 mM NaCl, and 10 mM imidazole. Once the loading was complete the column was washed with a wash buffer containing 50 mM Na₃PO₄, 20 mM NaCl, and 10 mM imidazole and a second wash buffer containing 50 mM Na₃PO₄, 20 mM NaCl, 10 mM imidazole, and 0.1% Triton X-114 to remove endotoxin. Once the washes were complete the His-tagged protein was eluted with 14.5 ml of 50 mM Na₃PO₄, 20 mM NaCl, and 400 mM imidazole. The flow rate for the conditioned media, wash buffer, and elution buffer through the Ni Excel column was set to 5 ml/min.

A desalting column (Cytiva HiPrep™ 26/10, with bed volume of 53 ml and a 1000-5000 molecular weight cut-off) was used for buffer exchanging before loading the His-tagged protein that eluted from the Ni Excel column onto the Ni NTA column. Due to the high salt content of the Ni Excel eluate, it could not be loaded directly onto the Ni NTA column. The desalting column was a HiPrep™ 26/10 desalting column (Cytiva, Cat. #17-5087-01). A 50 ml desalting column used in this method has the capacity to buffer exchange up to 15 ml of protein solution. A 10 ml loop captured the elution from the Ni Excel column and from there loaded over the desalting column. The flow rate set for the desalting column was 10 ml/min.

The buffer exchanged protein coming out of the desalting column was collected in a 20 ml loop and loaded from it onto a 5 ml Ni NTA Superflow™ column pre-equilibrated with 50 mM Na₃PO₄, 20 mM NaCl, and 10 mM imidazole. Once the loading was complete the column was washed with 50 mM Na₃PO₄, 20 mM NaCl, and 10 mM imidazole to remove and with 50 mM Na₃PO₄, 20 mM NaCl, 10 mM imidazole, and 0.1% Triton X-114 to remove endotoxin. Once the washes were complete the His-tagged protein was eluted with 15 ml of 50 mM Na₃PO₄, 20 mM NaCl, and 400 mM imidazole. The flow rate for the conditioned media, wash buffer, and elution buffer through the Ni NTA column was set to 5 ml/min. The Ni NTA Superflow™ elution was collected into a 7 ml loop and loaded from there onto a Superdex (SEC) column.

Both Nickel columns were regenerated after every run using 0.5 M NaOH and the sample pump was also washed after every run. This system can queue in the purification of several expressions at the time in a hands free fully automated mode without considering levels of expression.

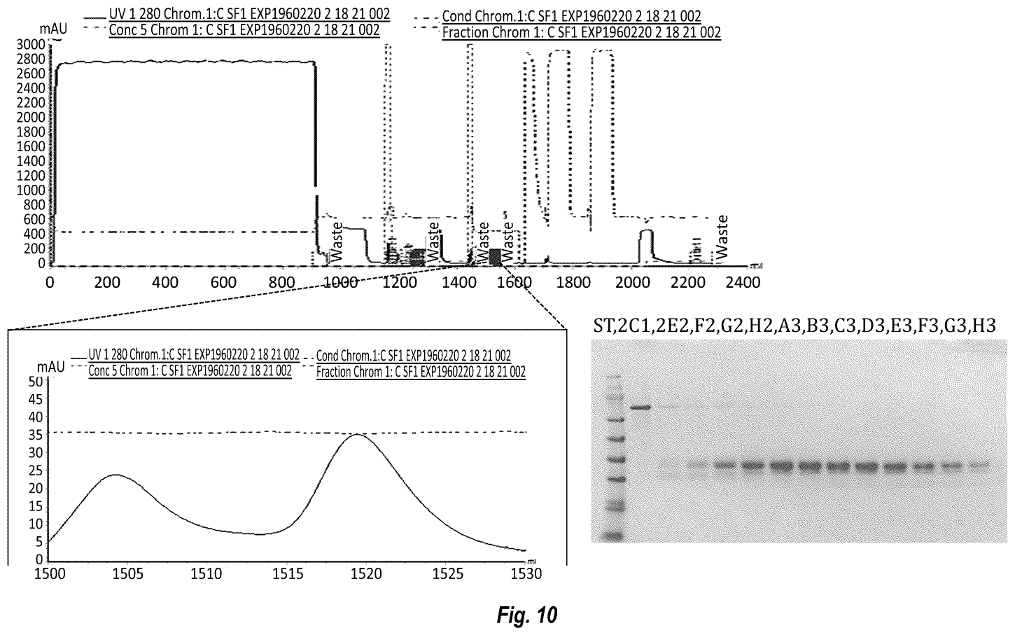

FIG. 9 shows a diagram of this system. FIG. 10 shows that a His-tagged polypeptide was purified with high yield using an embodiment of the method described in the present application. The polypeptide was purified to greater than 95% purity, even though the expression level in the cell culture was low. Without the tandem of Ni-Excel+Ni-NTA column purification method used here, it may not have been possible to purify this low-expressing polypeptide to such a high degree of purity in a high throughput manner. The same method described above was performed except with a polypeptide purified from a CHO media instead of BEVS media.

### Example 5: Four Column Purification of a Protein Expressed in Insect Cells

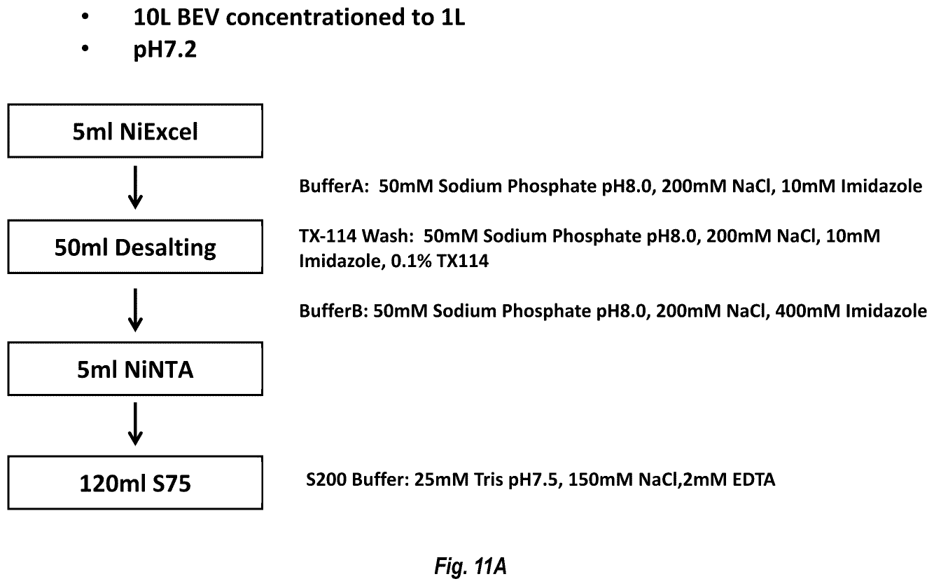

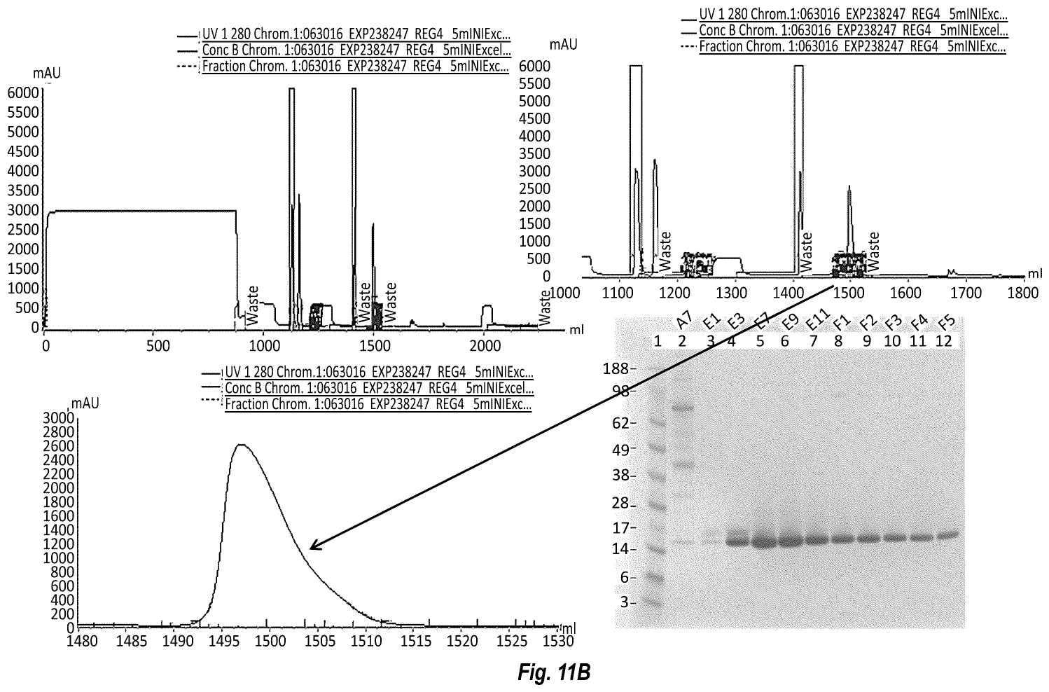

In a further example, a protein in BEVS media was purified using a 5 mL Ni Excel column (Cytiva, Cat #17371201), desalting column, 5 mL Ni NTA column (Qiagen, Cat #30761), and a 120 mL Superdex® 75 (SEC) column. A 10 liter cell lysate in BEVS buffer at pH 7.2 was concentrated to 1 liter. The concentrated protein in BEVS buffer was loaded onto the 5 mL NiExcel column pre-equilibrated in 50 mM sodium phosphate pH 8.0, 200 mM NaCl, and 10 mM imidazole. The column was washed with 50 mM sodium phosphate pH 8.0, 200 mM NaCl, 10 mM imidazole, and 0.1% Triton X114 before eluting the protein. The protein was eluted in 50 mM sodium phosphate pH 8.0, 200 mM NaCl, and 400 mM imidazole. After elution, the protein was subjected to desalting in a 50 mL volume desalting column so that it could be loaded onto a NiNTA column. The desalted solution was then loaded onto the 5 mL NiNTA column using the same equilibration, wash, and elution buffers as above. The NiNTA column eluate was then further purified by size exclusion chromatography (SEC) on a 120 mL Superdex® 75 column in 25 mM Tris pH 7.5, 150 mM NaCl, and 2 mM EDTA. (See FIG. 11A.) FIG. 11B shows results of the batch purification process. The protein was purified to a concentration of 2.9 mg/mL (specifically 31.9 mg in a volume of 11 mL) and had an endotoxin concentration of 0.08 U/mg. As shown by the gel in FIG. 11B, the protein was highly pure following this process.

### Example 6: Four Column Purification of a Protein Expressed in CHO Cells

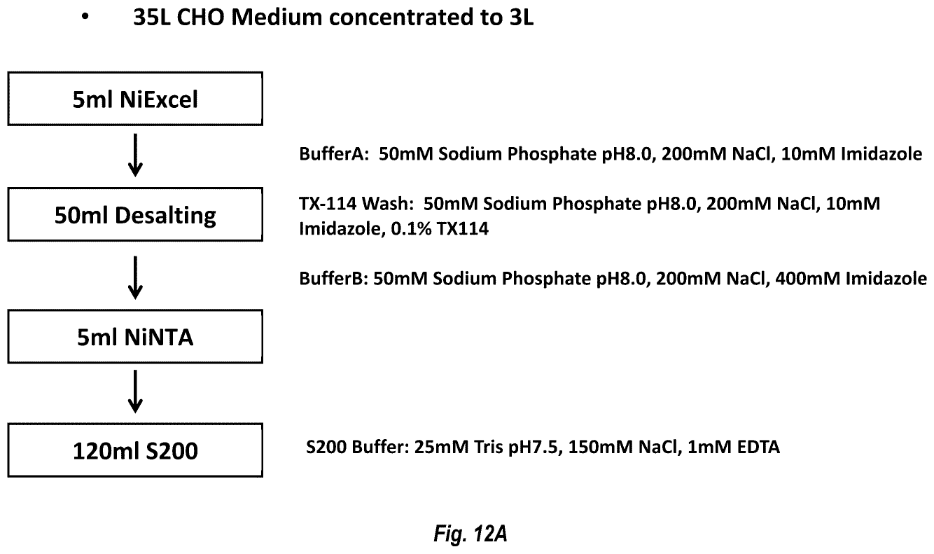

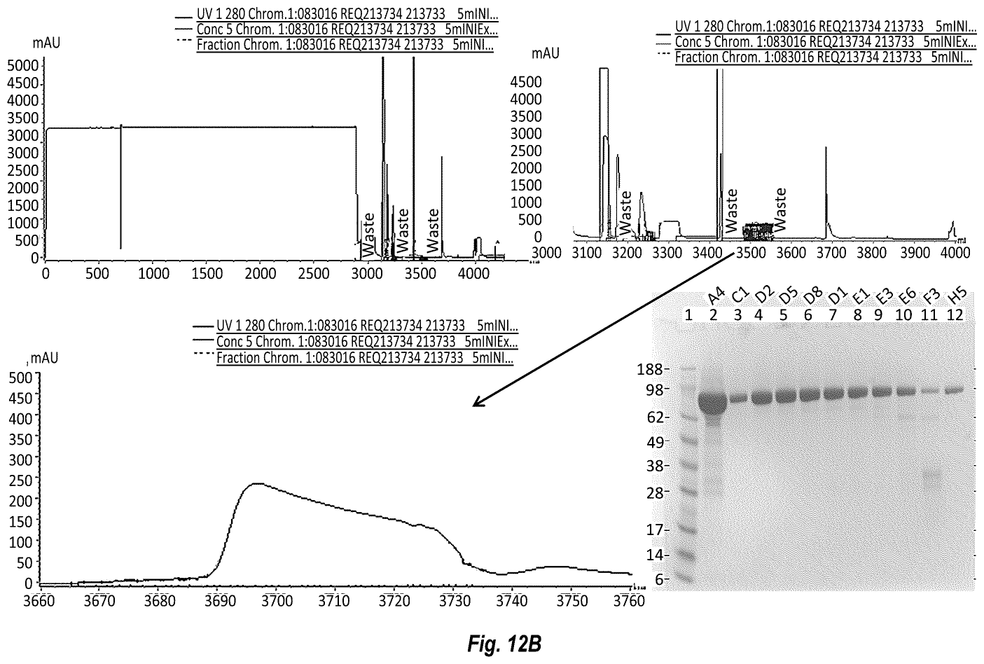

In a further experiment, an additional protein was expressed in CHO cells. A 35 liter volume of protein in CHO buffer was concentrated to 3 liters, and loaded in batches into a four column system comprising a 5 mL NiExcel column, a 50 mL desalting column, a 5 mL NiNTA column, and a Superdex® 200 column. The concentrated protein in CHO buffer was loaded in batches onto the 5 mL NiExcel column pre-equilibrated in 50 mM sodium phosphate pH 8.0, 200 mM NaCl, and 10 mM imidazole. The column was washed with 50 mM sodium phosphate pH 8.0, 200 mM NaCl, 10 mM imidazole, and 0.1% Triton X114 before eluting the protein. The protein was eluted in 50 mM sodium phosphate pH 8.0, 200 mM NaCl, and 400 mM imidazole. After elution, the protein was subjected to desalting in a 50 mL volume. The desalted solution was then loaded onto the 5 mL NiNTA column using the same equilibration, wash, and elution buffers. The NiNTA column eluate was then further purified by size exclusion chromatography (SEC) on a 120 mL Superdex® 200 column in 25 mM Tris pH 7.5, 150 mM NaCl, and 1 mM EDTA. (See FIG. 12A.) FIG. 12B shows the results of the purification, resulting in a protein concentration of 0.67 mg/mL (16 mL with 10.7 mg protein).

What is claimed is:

1. A method for purifying a polypeptide, comprising:
(a) loading a composition comprising the polypeptide onto a first immobilized metal affinity chromatography (IMAC) matrix;
(b) eluting the polypeptide from the first IMAC matrix with an elution buffer to form a first eluate comprising the polypeptide;
(c) conducting a buffer exchange to remove the elution buffer from the first eluate;
(d) loading the first eluate from step (c) onto a second IMAC matrix; and
(e) eluting the polypeptide from the second IMAC matrix with a second elution buffer to form a second eluate comprising the polypeptide.

2. The method of claim 1, further comprising conducting size exclusion chromatography (SEC) on the second eluate.

3. The method of claim 1 or 2, further comprising detecting the polypeptide in the first or second eluate or following SEC, optionally by electrophoresis or mass spectrometry.

4. The method of any one of claims 1-3, wherein the first IMAC matrix comprises agarose beads and a chelating ligand bound to nickel.

5. The method of any one of claims 1-4, wherein the first IMAC matrix retains nickel when exposed to cell culture media.

6. The method of any one of claims 1-5, wherein the second IMAC matrix comprises agarose beads and a nitrilotriacetic acid chelation moiety bound to nickel.

7. The method of any one of claims 1-6, further comprising regenerating the first IMAC matrix and/or the second IMAC matrix by washing with a regeneration buffer.

8. The method of claim 7, wherein the regeneration buffer comprises sodium hydroxide.

9. The method of any one of claims 1-8, wherein the polypeptide comprises a polyhistidine tag (His-Tag).

10. The method of any one of claims 1-9, wherein the polypeptide is an antibody, cell receptor, intracellular protein, secreted protein, or membrane protein.

11. The method of any one of claims 1-10, wherein the buffer exchange comprises passing the first eluate over a desalting column.

12. The method of any one of claims 1-11, wherein the first elution buffer and the second elution buffer are the same.

13. The method of any one of claims 1-12, wherein the composition comprises cell culture media.

14. The method of claim 13, wherein the cell culture media is conditioned cell culture media.

15. The method of any one of claims 1-14, further comprising washing the first IMAC matrix with a wash buffer after step (a) and before step (b).

16. The method of any one of claims 1-15, further comprising washing the second IMAC matrix with a wash buffer after step (d) and before step (e).

17. The method of any one of claims 1-16, wherein a controller directs flow through columns that contain the first IMAC matrix and the second IMAC matrix the method is automated.

18. The method of any one of claims 1-17, further comprising determining an elution time window for the first eluate and the second eluate.

19. A polypeptide which has been purified according to the method of any one of claims 1-18.

20. A system for purifying a polypeptide, wherein the system comprises:
(a) an injection valve for injecting a composition containing the polypeptide into the system, wherein the injection valve is connected to a first immobilized metal affinity chromatography (IMAC) matrix and a pump to control flow of the sample through the first IMAC matrix;
(b) a column valve for controlling flow path of an equilibration buffer, an elution buffer, and an SEC buffer;
(c) a desalting column configured to receive flowthrough or eluate from the first IMAC matrix;
(d) a second IMAC matrix configured to receive flowthrough or eluate from the desalting column;
(e) a size exclusion chromatography (SEC) column configured to receive flowthrough or eluate from the second IMAC matrix; and
(f) a detection device for detecting the polypeptide in the system.

21. The system of claim 20, wherein the injection valve and the column valve can be automatically controlled.

22. The system of claim 20 or 21, wherein the first and/or second IMAC matrix is an IMAC column.

23. The system of any one of claims 20-22, wherein the detection device is a UV spectrometer or mass spectrometer.

24. A method for purifying a His-tagged polypeptide, comprising:
(a) loading a composition comprising the His-tagged polypeptide onto a first immobilized metal affinity chromatography (IMAC) matrix;
(b) eluting the His-tagged polypeptide from the first IMAC matrix with an elution buffer to form a first eluate comprising the polypeptide;
(c) conducting a buffer exchange to remove the elution buffer from the first eluate using a desalting column;
(d) loading the first eluate from step (c) onto a second IMAC matrix, wherein the second IMAC matrix comprises agarose beads and a nitrilotriacetic acid chelation moiety bound to nickel; and
(e) eluting the His-tagged polypeptide from the second IMAC matrix with a second elution buffer to form a second eluate comprising the His-tagged polypeptide.

25. The method of claim 24, further comprising conducting size exclusion chromatography (SEC) on the second eluate to collect purified polypeptide.

26. The method of claim 25, further comprising detecting the purified polypeptide collected from the SEC, optionally by electrophoresis or mass spectrometry.

27. The method of any one of claims 24-26, wherein the first IMAC matrix comprises agarose beads and a chelating ligand bound to nickel.

28. The method of any one of claims 24-27, wherein the first IMAC matrix retains nickel when exposed to cell culture media.

29. The method of any one of claims 24-28, wherein the second IMAC matrix comprises agarose beads and a nitrilotriacetic acid chelation moiety bound to nickel.

30. A polypeptide which has been purified according to the method of any one of claims 24-29.
